(19) United States
(12) Patent Application Publication
(10) Pub. No.: US 2019/0088359 A1
(43) Pub. Date: Mar. 21, 2019

(54) System and Method for Automated Analysis in Medical Imaging Applications

(71) Applicant: Geisinger Health System, Danville, PA (US)

(72) Inventors: Gregory J. Moore, Danville, PA (US); Aalpen A. Patel, Danville, PA (US); Andrew M. Michael, Danville, PA (US); Mohammad R. Arbabshirani, Danville, PA (US)

(21) Appl. No.: 16/080,808

(22) PCT Filed: Mar. 3, 2017
(86) PCT No.: PCT/US17/20780

§ 371 Date: Aug. 29, 2018

Related U.S. Application Data
(60) Provisional application No. 62/303,070, filed on Mar. 3, 2016.
(60) Provisional application No. 62/334,900, filed on May 11, 2016.

(51) Int. Cl. G16H 30/40; G06F 17/30; G16H 15/00; G06N 3/04; G06T 7/00; G06K 9/62

(52) U.S. Cl. CPC G16H 30/40; G06F 17/30256; G16H 15/00; G06N 3/04; G06T 7/0012; G06K 2209/05; G06T 2207/20076; G06T 2207/30052; G06T 2207/20084; G06T 2207/20081; G06T 2207/20092; G06K 9/6228

(57) ABSTRACT

Systems and methods configured for providing automated analysis of images in medical imaging applications are disclosed. A system configured in accordance with the present disclosure may include an analyzer configured to recognize a feature of interest in a medical image utilizing a predictive model developed at least partially based on machine learning of previously acquired medical data. The analyzer may also be configured to determine whether the feature of interest is an abnormality or a hardware implant. The system configured in accordance with the present disclosure may help increase efficiency of radiology practice and disease detection rates.

### TECHNICAL FIELD

The present disclosure generally relates to the field of medical image analysis, and particularly to systems and methods for automated analysis in medical imaging applications.

### BACKGROUND

Medical image analysis is a medical specialty that uses imaging to diagnose and treat diseases. It is noted that imaging techniques such as X-ray radiography, ultrasound, computed tomography (CT), nuclear medicine including positron emission tomography (PET), magnetic resonance imaging (MRI) and the like can be very useful in diagnosing and treating diseases.

It is also noted, however, that health care professionals (e.g., radiologists) may have very limited time available to review the various medical images provided to them due to the amount of patients received and the high number of images produced per patient. Health care professionals may also introduce errors that negatively affect the accuracy of the analysis results. Therein lies a need to help reduce the workload of the health care professionals and improve the accuracy of the analysis.

### SUMMARY

An embodiment of the present disclosure is directed to a method. The method may include: receiving a medical image of a patient; recognizing a feature of interest in the medical image of the patient utilizing a predictive model developed at least partially based on machine learning of previously recorded medical data; determining whether the feature of interest is an abnormality utilizing the predictive model; and reporting a probability of the feature of interest being an abnormality to a user.

A further embodiment of the present disclosure is directed to a method. The method may include: receiving a medical image of a patient; recognizing a feature of interest in the medical image of the patient utilizing a predictive model developed at least partially based on machine learning of previously recorded medical data; determining whether the feature of interest is a hardware implant inside the patient; and reporting an identification of the hardware implant to a user.

An additional embodiment of the present disclosure is directed to a system. The system may include an imaging device configured to acquire a medical image of a patient. The system may also include a data storage device in communication with the imaging device. The data storage device may be configured to store the medical image of the patient and previously acquired medical data. The system may further include an analyzer in communication with the data storage device. The analyzer may be configured to: recognize a feature of interest in the medical image of the patient utilizing a predictive model developed at least partially based on machine learning of the previously acquired medical data; determine whether the feature of interest is an abnormality or a hardware implant inside the patient utilizing the predictive model; and report a determined result to a user.

It is to be understood that both the foregoing general description and the following detailed description are exemplary and explanatory only and are not necessarily restrictive of the present disclosure. The accompanying drawings, which are incorporated in and constitute a part of the specification, illustrate subject matter of the disclosure. Together, the descriptions and the drawings serve to explain the principles of the disclosure.

### BRIEF DESCRIPTION OF THE DRAWINGS

The numerous advantages of the disclosure may be better understood by those skilled in the art by reference to the accompanying figures in which:

FIG. 1 is a block diagram depicting a medical imaging analysis system configured in accordance with the present disclosure;

FIG. 2 is a flow diagram depicting a medical imaging analysis method configured in accordance with the present disclosure;

FIG. 3 is a flow diagram depicting a method for developing a model suitable for processing medical images;

FIG. 4 is an illustration depicting an exemplary convolutional neural network created for medical image analysis;

FIG. 5 is a flow diagram depicting an exemplary radiology workflow in accordance with the present disclosure; and

FIG. 6 is an illustration depicting an exemplary report provided in accordance with the present disclosure.

### DETAILED DESCRIPTION

Reference will now be made in detail to the subject matter disclosed, which is illustrated in the accompanying drawings.

Embodiments in accordance with the present disclosure are directed to systems and methods configured to provide automated analysis of images in medical imaging applications. Various machine learning and data analytic techniques may be implemented in systems configured in accordance with the present disclosure to help increase efficiency and disease detection rates, which may in turn reduce costs and improve quality of care for patients.

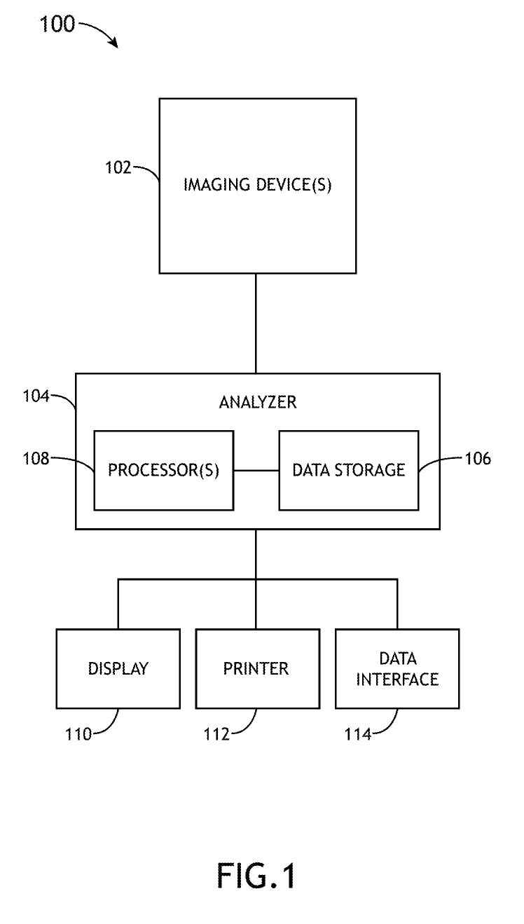

Referring generally to FIG. 1, a block diagram depicting a medical imaging analysis system 100 configured in accordance with the present disclosure is shown. The medical imaging analysis system 100 may include one or more medical imaging devices 102 (e.g., X-ray radiography devices, ultrasound imaging devices, CT imaging devices, tomography PET devices, MRI devices, cardiac imaging devices, digital pathology devices, endoscopy devices, arthoscopy devices, medical digital photography devices, ophthalmology imaging devices or the like) in communication with an analyzer 104. The analyzer 104 may include one or more data storage devices 106 (e.g., magnetic storage devices, optical storage devices, solid-state storage devices, network-based storage devices or the like) configured to store images acquired by the medical imaging devices 102.

The one or more data storage devices 106 may be further configured to serve as a data repository of historical data, which may form a large data set that may be referred to as “big data.” This large data set can be utilized to train one or more predictive models executed on one or more processing units 108 of the analyzer 104. The predictive model(s) trained in this manner may then be utilized to analyze images acquired by the medical imaging devices 102. For example, the analyzer 104 may be configured to recognize one or more features of interest in the images acquired by the medical imaging devices 102. The analyzer 104 may also be configured to determine whether the feature/features of interest represent abnormality/abnormalities. The analyzer 104 may be further configured to determine whether a feature of interest is a certain type of object (e.g., a hardware implant) inside a patient's body. Determinations made by the analyzer 104 may be recorded (e.g., into the data storage devices 106) and/or reported to a user (e.g., a radiologist, a doctor or the like) via one or more output devices 110-114.

The one or more processors 108 of analyzer 104 may include any one or more processing elements known in the art. In this sense, the one or more processors 108 may include any microprocessor device configured to execute algorithms and/or instructions. For example, the one or more processors 108 may be implemented within the context of a desktop computer, workstation, image computer, parallel processor, mainframe computer system, high performance computing platform, supercomputer, or other computer system (e.g., networked computer) configured to execute a program configured to operate the system 100, as described throughout the present disclosure. It should be recognized that the steps described throughout the present disclosure may be carried out by a single computer system or, alternatively, multiple computer systems. In general, the term “processor” may be broadly defined to encompass any device having one or more processing elements, which execute program instructions from a memory medium (e.g., data storage device 106 or other computer memory). Moreover, different subsystems of the system 100 (e.g., imaging device 102, display 110, printer 112 or data interface 114) may include processor or logic elements suitable for carrying out at least a portion of the steps described throughout the present disclosure. Therefore, the above description should not be interpreted as a limitation on the present invention but merely an illustration.

The one or more data storage device 106 may include any data storage medium known in the art suitable for serving as a data repository of historical data and/or storing program instructions executable by the associated one or more processors 108. For example, the one or more data storage devices 106 may include a non-transitory memory medium. For instance, the one or more data storage device 106 may include, but are not limited to, a read-only memory, a random access memory, a magnetic or optical memory device (e.g., disk), a magnetic tape, a solid state drive and the like. It is further noted that one or more data storage device 106 may be housed in a common controller housing with the one or more processors 108. In another embodiment, the one or more data storage device 106 may be located remotely with respect to the physical location of the one or more processors 108 and analyzer 104. For instance, the one or more processors 108 of analyzer 104 may access a remote memory (e.g., server), accessible through a network (e.g., internet, intranet and the like). The one or more data storage device 106 may store the program instructions for causing the one or more processors 104 to carry one or more of the various steps described through the present disclosure.

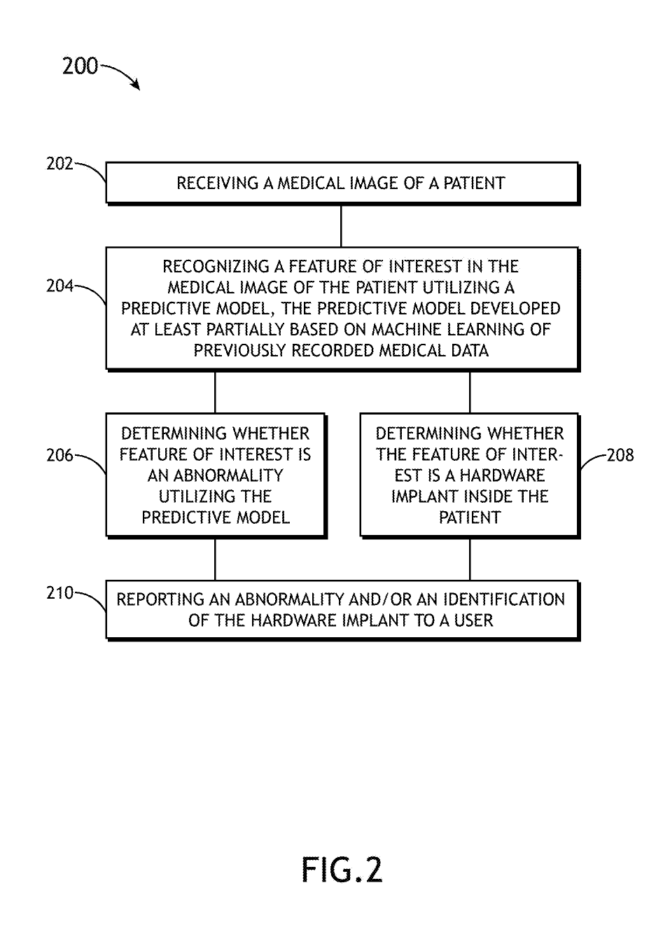

Referring now to FIG. 2, a flow diagram depicting a medical imaging analysis method 200 configured in accordance with the present disclosure is presented. As shown in FIG. 2, upon receiving a medical image (e.g., a radiological image) of a patient in a step 202, a predictive model developed at least partially based on machine learning of previously recorded medical data may be utilized in a step 204 to recognize one or more features of interest in the newly received medical image. The predictive model may also be utilized to determine whether a feature of interest represents an abnormality in a step 206, and/or whether a feature of interest represents a hardware implant inside the patient in a step 208. The probability of a feature of interest being an abnormality or a hardware implant may then be reported to a user in a step 210 using one or more output devices.

In some embodiments, the output devices may include one or more electronic displays. Alternatively and/or additionally, the output devices may include printers or the like. In some embodiments, the output devices may include electronic interfaces such as computer display screens, web reports or the like. In such embodiments, analysis results (e.g., reports) may be delivered via electronic mails and/or other electronic data exchange/interchange systems without departing from the spirit and scope of the present disclosure.

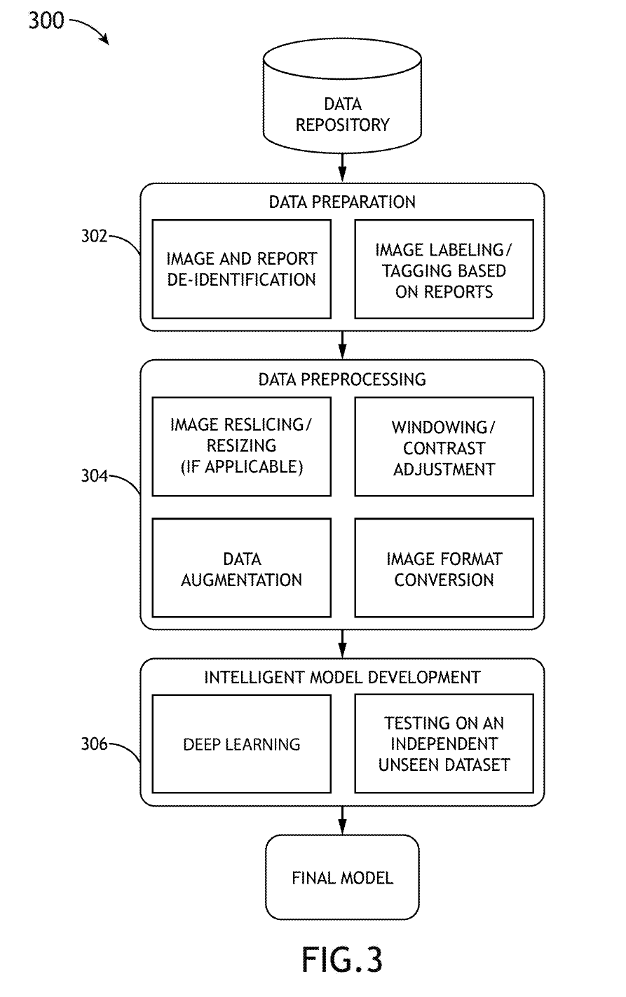

It is noted that both the medical imaging analysis system 100 and the medical imaging analysis method 200 described above referenced predictive models developed using machine learning. FIG. 3 is a flow diagram depicting an exemplary method 300 for developing such a predictive model. In some embodiments, the predictive model may be developed/trained at least partially based on medical data (e.g., images and reports) retrieved from a data repository (e.g., from a Picture Archiving and Communication System, or PACS) or other archives. In some embodiments, if protected health information (e.g., patient-identifying information or the like) is present along with the medical data retrieved, a data preparation step 302 may be invoked to perform a process referred to as de-identification, which helps remove protected health information from the data retrieved. In some embodiments, information such as demographics and the like may be kept after de-identification. The data preparation step 302 may also extract clinically relevant labels (e.g., normal or abnormal) and/or abnormality tags (e.g., brain atrophy or the like) form the reports associated with the medical images. It is contemplated that labels and/or tags extracted in this manner may help training the predictive model, providing the predictive model with capabilities of detecting potential abnormalities in new images.

In some embodiments, the medical images retrieved from the data repository may undergo one or more data preprocessing operations in a data preprocessing step 304. It is to be understood that the data preprocessing operations depicted in the data preprocessing step 304 are presented merely for illustrative purposes and are not meant to be limiting. For example, images recorded in different formats may be extracted and converted to a common format. Images of different sizes may be resized (e.g., in the X and Y directions) and/or resliced (e.g., in the Z direction for 3D images) to a predetermined size. Additional image enhancement techniques, such as contrast adjustment for certain images (e.g., windowing for CT images), may also be applied to make certain abnormalities more readily identifiable. In still another example, data augmentation techniques, including artificially increasing the sample size by creating multiple instances of the same image using operations such as rotation, translation, mirroring, and changing reslicing parameters, may also be employed in the data preprocessing step 304, along with other optional data preprocessing techniques without departing from the spirit and scope of the present disclosure.

The data prepared in this manner may be utilized to help train one or more predictive models in a model development step 306. The predictive model(s) may execute on one or more processing units (e.g., graphical processing units, or GPUs, central processing units, or CPUs), and a training process that uses machine learning may be utilized to train the predictive model(s) based on the prepared data. Suitable machine learning techniques may include, for example, a convolutional neural network (CNN), which is a type of feed-forward artificial neural network that uses multiple layers of small artificial neuron collections to process portions of the training images to build a predictive model for image recognition. The CNN architecture may include one or more convolution layers, max pooling layers, contrast normalization layers, fully connected layers and loss layers. Each of these layers may include multiple parameters. It is noted that while some of the parameters are utilized to govern the entire training process (including parameters such as choice of loss function, learning rate, weight decay coefficient, regularization parameters and the like), values applied to other parameters may be changed (e.g., CCN parameters and layers trained on head CT images may need to be changed for chest X-ray images), which may in turn change the CNN. The CNN may also be changed when the number, the size, and/or the sequence of its layers are changed, allowing the CNN to be trained accordingly.

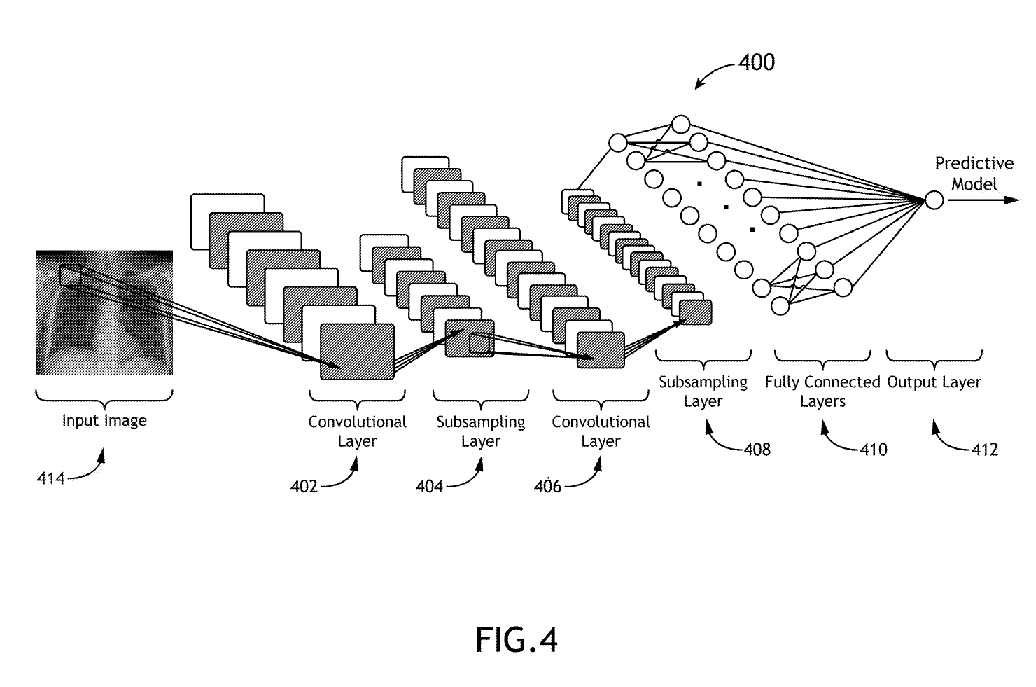

FIG. 4 shows an exemplary CNN 400 created for medical image analysis purposes. The exemplary CNN 400 may include two convolutional layers 402 and 406, two subsampling (pooling) layers 404 and 408, two fully connected layers 410, and an output node 412. Training of the CNN 400 may start with assigning (usually random) initial values to the various parameters used in the various layers 402-412. Batches of training images (e.g., chest radiographs and their labels) may then be provided as input 414 to the CNN 400, which may prompt the values of the various parameters to be updated in iterations based on the changes in the loss function value. The training process may continue until the model converges and the desired output is achieved.

It is to be understood that the CNN 400 described above is merely exemplary and is not meant to be limiting. It is contemplated that the number of layers in a CNN may differ from that described above without departing from the spirit and scope of the present disclosure. It is also to be understood that while a training process using a feed-forward artificial neural network is described in the example above, such descriptions are merely exemplary and are not meant to be limiting. It is contemplated that other types of artificial neural networks, including recurrent neural networks (RNN) and the like, as well as various other types of deep learning techniques (e.g., machine learning that uses multiple processing layers), may also be utilized to facilitate the machine learning process without departing from the spirit and scope of the present disclosure.

It is also contemplated that the training process may be carried out iteratively. In some embodiments, a testing step may be utilized to measure the accuracy of the predictive model(s) developed. Testing may be performed using dataset(s) and image(s) from the data repository that are not used for training. If the accuracy of a predictive model is deemed satisfactory (e.g., the accuracy is above a certain threshold), the training process may be considered complete and the predictive model may be utilized to process/analyze new images.

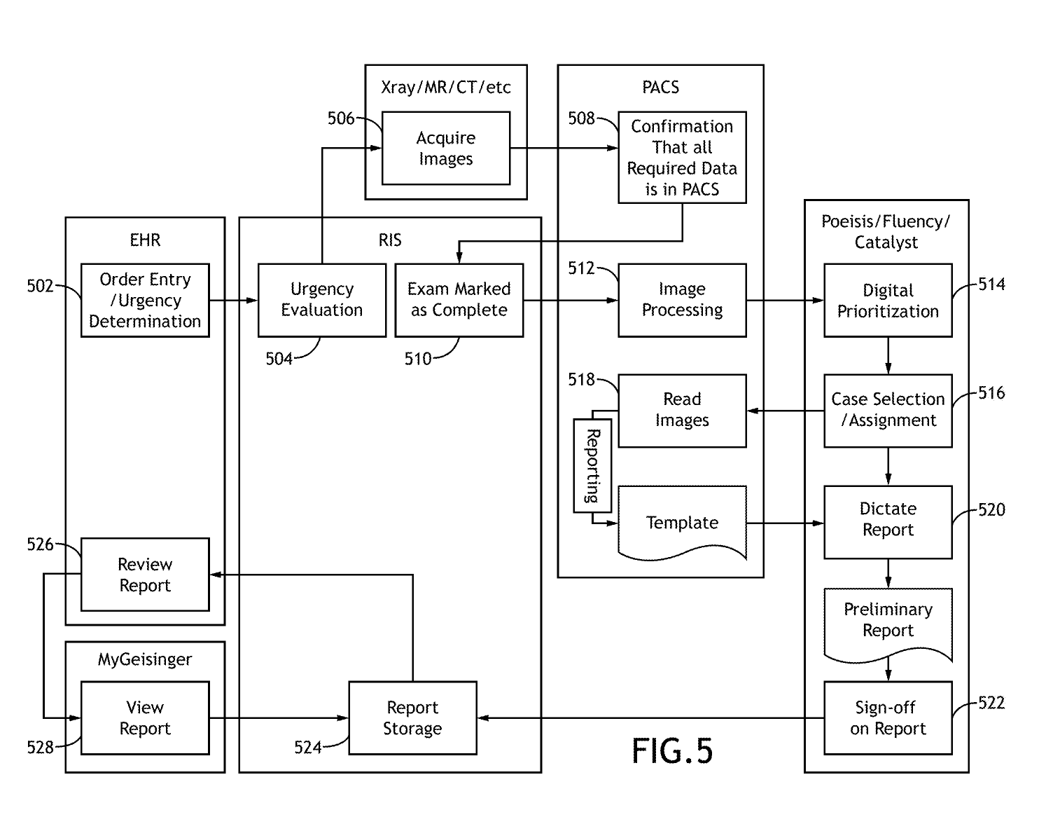

Referring generally to FIG. 5, a more detailed flow diagram depicting a workflow 500 using image recognition and predictive modeling techniques configured in accordance with the present disclosure is shown. In the example depicted in FIG. 5, health information of a patient may be entered (manually and/or systematically) into an Electronic Health Record (EHR) in a step 502. The information entered may be provided to a Radiology Information System (RIS), which may include a networked software system for managing medical imagery and associated data. The patient may be evaluated in a step 504 and medical exam(s) and/or image(s) needed for the patient may be determined and subsequently acquired in a step 506. The exam data and the acquired images may be provided to a data repository (e.g., a Picture Archiving and Communication System, or PACS) in a step 508, and once all required information is received in the data repository, the exam may be marked as complete in a step 510 and one or more previously trained predictive models may be utilized to analyze the received information (e.g., the exam data and the medical images) in a step 512.

For example, a predictive model may be utilized to recognize certain features in the acquired images and to stratify/flag the images based on criticalities of the features recognized. More specifically, following completion of a medical exam for a patient, the medical images obtained for that patient may be preprocessed and fed to a predictive model for analysis. If no abnormality is recognized by the predictive model, the analysis result may be considered “normal”, and a report may be generated for a radiologist to confirm. On the other hand, if one or more abnormalities are detected, the abnormalities may be identified in the report.

In some implementations, the report may include a value indicating the probability that the analysis result is “normal” or “abnormal”, which may be made available to health care professionals (e.g., radiologists) as a reference. In some implementations, if the probability exceeds certain threshold established for abnormality, further analysis (e.g., using alternative/additional imaging systems and/or performing follow-up studies by radiologists) may be scheduled and/or prioritized for that patient in a step 514. It is noted that utilizing the predictive model to perform analysis in this manner may help reduce the risk of oversight and may help facilitate assignment of the patient to appropriate health care professionals in a step 516 based on the nature and criticality of the abnormalities detected, which in turn may improve the quality of patient care provided.

It is also noted that the health care professionals utilizing the predictive model may help refine the predictive model through actions taken by the health care professionals. For example, if a health care professional (e.g., a radiologist) modifies a report generated using a predictive model from “normal” to “abnormal”, this modification may be fed back (e.g., via a feedback mechanism) to the predictive model, allowing the predictive model to learn from its inaccurate predictions. In another example, if the predictive model mistakenly prioritizes a patient due to a misidentified abnormality, a health care professional may correct the mistake and allow the predictive model to learn from its mistakes. It is noted that the feedback mechanism may also be utilized to communicate to the predictive model regarding predictions confirmed by the health care professionals, allowing the predictive model to affirm its correct predictions.

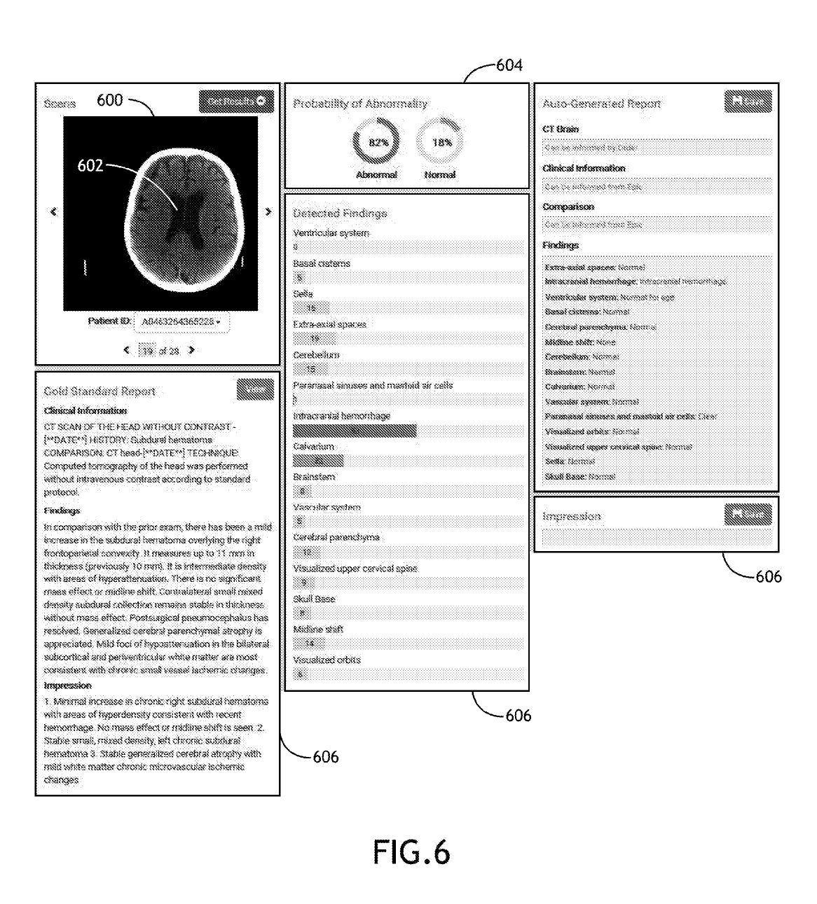

It is further noted that while the aforementioned report may be presented as a text-based report, some embodiments in accordance with the present disclosure may be configured to generate a more informative, text- and/or graphic-based report depicted in FIG. 6. As shown generally in FIGS. 5 and 6, a predictive model developed based on a repository of CT images may process a CT image 600 of a patient in a step 518 and recognize that the CT image 600 of the patient exhibits a feature 602 that may be of an interest. The predictive model may also determine that there is a certain probability 604 for the feature 602 to be considered abnormal because the feature 602 is likely to represent a mild increase in the subdural hematoma overlaying the right frontoparietal convexity. Findings as such may be utilized to pre-populate certain fields on a report template and may be provided to radiologists for review. Optional and/or additional support information 606 may also be provided to help radiologists make more informed decisions.

It is to be understood that while the report shown in FIG. 6 may be generated based on certain reporting standards and/or templates, such standards and/or templates are merely exemplary and are not meant to be limiting. It is contemplated that the format of the report may vary from the illustration depicted in FIG. 6 without departing from the spirit and scope of the present disclosure. Regardless of the specific format, however, it is noted that providing the abilities to automatically generate at least some portions of the medical report may help reduce the amount of time radiologists may otherwise have to spend (in a step 520) on preparing such a report. While radiologists using automatically generated reports may still need to review the reports in a step 522 and make necessary changes and/or additions, the amount of work required may be significantly reduced using an automated process, which in turn may help reduce the cost of medical studies.

It is noted that medical reports produced in this manner may be provided to an information system (e.g., a radiology information system) in a step 524, allowing the information provided in the reports to be selectively accessible to patient as well as other users (e.g., doctors and family members) in a step 528. It is contemplated that the format of the report and the amount of information accessible to each user viewing the report may vary (e.g., certain information may be made accessible only to the patient's doctor). It is therefore contemplated that optional/additional report processing steps 526 may be invoked to help adjust the format of the report and filter the information included in each report.

It is further contemplated that a report produced in accordance with the present disclosure is not limited to merely indicating whether an image contains any abnormalities. For instance, in certain implementations, a predictive model may be trained to recognize certain types of hardware (e.g., a pacemaker or the like) that may be implanted inside the patient. For example, CNN may be used to train a predictive model using training images in a manner similar to that described above. Alternatively and/or additionally, computer vision techniques such as speeded-up robust features (SURF), histogram of oriented gradients (HoG), GIST descriptors and the like may be used to extract comprehensive feature vectors to represent the training images, allowing a classifier (e.g., a non-linear support vector machine classifier) to be used to build predictive models capable of detecting presence of certain types of hardware in the training images. In case of pacemakers, for example, if a pacemaker is detected in an X-ray image obtained for a patient, further studies (e.g., chest MRI) that may cause complications to the patient due to the presence of the pacemaker may be flagged in the report to prompt further review. Such a report may help reduce risks to the patient and improve patient care efficiency.

It is contemplated that the predictive model training process may be fine tuned to support accurate detection of not only the presence of hardware implants, but also the type, make, and/or the model of the hardware in some implementations. It is also contemplated that the training images may be obtained from multiple angles to help create a more robust predictive model. It is to be understood that the level of detection provided may be determined based on various factors, including, but not limited to, data availability, time, as well as cost. It is to be understood that the level of detection provided may vary without departing from the spirit and scope of the present disclosure.

It is also to be understood that the depictions of CT images, head scans and chest X-rays referenced above are merely exemplary and are not meant to be limiting. It is contemplated that predictive models in accordance with the present disclosure may be developed based on a variety of image repositories using a variety of machine learning and data analytic techniques, and the predictive models developed in this manner may be configured to process a variety of medical images separately and/or jointly without departing from the spirit and scope of the present disclosure.

It is further contemplated that the predictive models developed in accordance with the present disclosure may be re-trained periodically, continuously, intermittently, in response to a predetermined event, in response to a user request or command, or combinations thereof. For example, user confirmations of (or modifications to) the analysis results provided by a predictive model may be recorded (e.g., in the RIS) and utilized as additional training data for the predictive model. It is contemplated that re-training of predictive models in this manner may help increase the accuracy and reduce potential errors (e.g., false positives and false negatives).

It is to be understood that the present disclosure may be conveniently implemented in forms of a software, hardware, or firmware package. It is also to be understood that embodiments of the present disclosure are not limited to any underlying implementing technology. Embodiments of the present disclosure may be implemented utilizing any combination of software and hardware technology and by using a variety of technologies without departing from the present disclosure or without sacrificing all of their material advantages.

It is to be understood that the specific order or hierarchy of steps in the processes disclosed is an example of exemplary approaches. It is to be understood that the specific order or hierarchy of steps in the processes may be rearranged while remaining within the broad scope of the present disclosure. The accompanying method claims present elements of the various steps in a sample order, and are not meant to be limited to the specific order or hierarchy presented.

It is believed that the systems and methods disclosed herein and many of their attendant advantages will be understood by the foregoing description, and it will be apparent that various changes may be made in the form, construction, and arrangement of the components thereof without departing from the broad scope of the present disclosure or without sacrificing all of their material advantages. The form herein before described being merely an explanatory embodiment thereof, it is the intention of the following claims to encompass and include such changes.

What is claimed is:

1. A method, comprising:
receiving a medical image of a patient;
recognizing a feature of interest in the medical image of the patient utilizing a predictive model, the predictive model developed at least partially based on machine learning of previously recorded medical data;
determining whether the feature of interest is an abnormality utilizing the predictive model; and
reporting a probability of the feature of interest being an abnormality to a user.

2. The method of claim 1, wherein said reporting step includes:
systematically populating at least a portion of a medical report for the user to confirm.

3. The method of claim 2, wherein said medical report includes a graphical representation of the feature of interest.

4. The method of claim 3, wherein said medical report further includes a text description of the feature of interest.

5. The method of claim 1, further comprising:
determining whether the patient needs an additional medical examination or evaluation at least partially based on the feature of interest.

6. The method of claim 5, further comprising:
prioritizing the additional medical examination or evaluation for the patient.

7. The method of claim 1, further comprising:
determining whether the feature of interest is a hardware implant inside the patient.

8. The method of claim 7, further comprising:
determining whether the patient is safe to undergo a medical examination or evaluation with a presence of the hardware implant inside the patient.

9. The method of claim 1, wherein development of the predictive model comprises:
creating a convolutional neural network having a plurality of layers;
assigning initial values to at least one parameter of at least one of the plurality of layers;
iteratively adjusting the at least one parameter of at least one of the plurality of layers based on recognition of a set of training images;
terminating the adjusting step when the convolutional neural network converges; and
providing the predictive model at least partially based on the converged convolutional neural network.

10. The method of claim 1, further comprising:
receiving an input from the user regarding the feature of interest; and
refining the predictive model at least partially based on the input received from the user.

11. A method, comprising:
receiving a medical image of a patient;
recognizing a feature of interest in the medical image of the patient utilizing a predictive model, the predictive model developed at least partially based on machine learning of previously recorded medical data;
determining whether the feature of interest is a hardware implant inside the patient; and
reporting an identification of the hardware implant to a user.

12. The method of claim 11, further comprising:
determining whether the patient is safe to undergo a medical examination or evaluation with a presence of the hardware implant inside the patient.

13. The method of claim 11, further comprising:
determining whether the feature of interest is an abnormality utilizing the predictive model; and
reporting a probability of the feature of interest being an abnormality to the user.

14. The method of claim 13, further comprising:
determining whether the patient needs an additional medical examination or evaluation at least partially based on the feature of interest.

15. The method of claim 14, further comprising:
prioritizing the additional medical examination or evaluation for the patient.

16. The method of claim 11, wherein said reporting step includes:
systematically populating at least a portion of a medical report for the user to confirm.

17. The method of claim 16, wherein said medical report includes a graphical representation of the feature of interest.

18. The method of claim 17, wherein said medical report further includes a text description of the feature of interest.

19. The method of claim 11, wherein development of the predictive model comprises:
creating a convolutional neural network having a plurality of layers;
assigning initial values to at least one parameter of at least one of the plurality of layers;
iteratively adjusting the at least one parameter of at least one of the plurality of layers based on recognition of a set of training images;
terminating the adjusting step when the convolutional neural network converges; and
providing the predictive model at least partially based on the converged convolutional neural network.

20. The method of claim 11, further comprising:
receiving an input from the user regarding the feature of interest; and
refining the predictive model at least partially based on the input received from the user.

21. A system, comprising:
a data storage device in communication with an imaging device configured to acquire a medical image of a patient, the data storage device configured to store the medical image of the patient and previously acquired medical data; and
an analyzer in communication with the data storage device, the analyzer configured to:
recognize a feature of interest in the medical image of the patient utilizing a predictive model, the predictive model developed at least partially based on machine learning of the previously acquired medical data;
determine whether the feature of interest is an abnormality or a hardware implant inside the patient utilizing the predictive model; and
report a determined result to a user.

22. The system of claim 21, wherein the analyzer is further configured to report a probability of the feature of interest being an abnormality to the user.

23. The system of claim 21, wherein the analyzer is further configured to report an identification of the hardware implant to a user.

24. The system of claim 21, wherein the analyzer is further configured to systematically populate at least a portion of a medical report for the user to confirm.

25. The system of claim 24, wherein said medical report includes a graphical representation of the feature of interest.

26. The system of claim 25, wherein said medical report further includes a text description of the feature of interest.

27. The system of claim 22, wherein the analyzer is further configured to determine whether the patient needs an additional medical examination or evaluation at least partially based on the feature of interest.

28. The system of claim 27, wherein the analyzer is further configured to prioritize the additional medical examination or evaluation for the patient.

29. The system of claim 22, wherein the analyzer is further configured to determine whether the patient is safe to undergo a medical examination or evaluation with a presence of the hardware implant inside the patient.

30. The system of claim 22, wherein the analyzer is further configured to develop the predictive model based on machine learning of the previously acquired medical data, wherein development of the predictive model comprises:
create a convolutional neural network having a plurality of layers;
assign initial values to at least one parameter of at least one of the plurality of layers;
iteratively adjust the at least one parameter of at least one of the plurality of layers based on recognition of a set of training images;
terminating adjustment of the at least one parameter when the convolutional neural network converges; and
provide the predictive model at least partially based on the converged convolutional neural network.

31. The system of claim 22, wherein the analyzer is further configured to receive an input from the user regarding the feature of interest and refine the predictive model at least partially based on the input received from the user.
